(19) United States
(12) Patent Application Publication
(10) Pub. No.: US 2025/0327797 A1
(43) Pub. Date: Oct. 23, 2025

(54) Birth Control Efficacy Monitoring Device

(71) Applicants: Zildjian Bryant, Katy, TX (US); Joycelyn Bryant, Katy, TX (US)

(72) Inventors: Zildjian Bryant, Katy, TX (US); Joycelyn Bryant, Katy, TX (US)

(21) Appl. No.: 18/668,404

(22) Filed: May 20, 2024

Related U.S. Application Data
(60) Provisional application No. 63/635,237, filed on Apr. 17, 2024.

(51) Int. Cl. G01N 33/543; G01N 33/68

(52) U.S. Cl. CPC G01N 33/54388; G01N 33/689; G01N 2333/575

(57) ABSTRACT

A personal birth control monitoring device designed to evaluate the effectiveness of hormonal birth control methods in females is disclosed. The device is a compact and portable kit and features an ergonomic, lightweight plastic casing with a semi-rigid test strip made of cotton or paper for urine absorption. The strip is protected by a removable lid to ensure sterility. Further, the strip is integrated with a flow assay strip which is pre-treated with chemicals reactive to birth control hormones, specifically estrogen and progesterone. The device includes a result window for immediate display of results, indicating the presence or absence of birth control hormones, thus reflecting the efficacy of the birth control pills taken by the user. The device provides a simple, quick, and user-friendly method for women to monitor the ongoing effectiveness of their hormonal birth control.

### CROSS-REFERENCE TO RELATED APPLICATION

The present application claims priority to, and the benefit of, U.S. Provisional Application No. 63/635,237, which was filed on Apr. 17, 2024, and is incorporated herein by reference in its entirety.

### FIELD OF THE INVENTION

The present invention generally relates to the field of birth control devices. More specifically, the present invention relates to a novel test kit for testing the levels of birth control hormones in female urine. The kit device looks similar to a pregnancy test but works to detect oestrogen and progesterone hormones that should be present while taking a contraception such as birth control pill. The device includes a strip of absorbing urine and a window for displaying test results. Accordingly, the present disclosure makes specific reference thereto. Nonetheless, it is to be appreciated that aspects of the present invention are also equally applicable to other like applications, devices, and methods of manufacture.

### BACKGROUND

By way of background, most females take birth control pills to prevent pregnancy as the birth control pill is a safe, simple, and convenient way to prevent pregnancy. Birth control pills contain estrogen and progesterone that prevent pregnancy by inhibiting ovulation. Estrogen and progestin in the pills work to stop the ovaries from releasing eggs. The hormones in birth control pills also thicken the mucus produced by the cervix. The thicker cervical mucus makes it more difficult for sperm to enter the uterus and reach any egg that might have been released.

However, birth control pills need to be taken daily to maintain consistent hormone levels for effective pregnancy prevention. Missing pills or taking them irregularly can lead to fluctuating hormone levels, reducing their effectiveness. There is no immediate or easy way for users to know if their hormone levels have been compromised due to missed or incorrectly taken pills.

Currently, the primary method to check for pregnancy prevention failure is a pregnancy test, which only indicates a failure after conception has already occurred. There is no proactive way to monitor the ongoing effectiveness of birth control pills. Many users of birth control pills may not have easy access to medical testing or may find it inconvenient or intimidating. People desire a simple, user-friendly device that enable them to check birth control hormone levels, specifically estrogen and progesterone at home like a conventional pregnancy kit.

Therefore, there exists a long felt need in the art for a birth control check device that monitors levels of estrogen and progesterone in urine. There is also a long felt need in the art for a monitoring kit that enables users to check if their hormone levels are consistent with effective birth control use. Additionally, there is a long felt need in the art for a novel birth control device that offers real-time feedback to people on the efficacy or use of their birth control method(s). Moreover, there is a long felt need in the art for a proactive birth control monitoring tool that enables users to potentially identify issues with their birth control regimen. Further, there is a long felt need in the art for a compact, portable, and easy-to-use device for monitoring of birth control effectiveness. Furthermore, there is a long felt need in the art for a handheld birth control device that displays results of birth control on a result window. Finally, there is a long felt need in the art for a test kit for testing the levels of birth control hormones such as estrogen and progesterone in female urine to help determine if the female is ovulating and birth control pill is ineffective.

The subject matter disclosed and claimed herein, in one embodiment thereof, comprises a birth control monitoring device for identifying efficacy of a birth control method or drug in a human body based on a urine sample obtained from the human body. The device includes a lightweight plastic casing extending between a first end and a second end, the casing is ergonomically designed for ease of handling, a test strip made of semi-rigid cotton or paper material (i.e., absorbent material) is attached at the second end of the casing, the test strip is configured for immersion in a urine stream for absorbing urine, a removable lid is removably engaged with the second end of the casing to protect the sterility of the test strip before use and a result window is disposed in the casing for displaying a result of the birth control check, wherein the result window is adapted to display different representations for a positive result and a negative result. An assay strip including estrogen detection and progesterone detection zone detects the birth control hormones in the absorbed urine sample.

In this manner, the birth control check device of the present invention accomplishes all of the forgoing objectives and provides users with a monitoring device to monitor hormone levels, specifically estrogen and progesterone, in urine of a subject. The device enables users to potentially identify issues with their birth control regimen before it leads to an unintended pregnancy. The device is compact, portable, and designed like a conventional pregnancy test kit for easy use. The device detects birth control hormones in the absorbed urine and displays results within two minutes of use, offering immediate feedback. The device potentially reduces the risk of unintended pregnancies due to inconsistent or incorrect use of birth control pills.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a birth control monitoring kit. The kit is an at-home hormone test kit designed to check and display whether birth control is working properly by detecting birth control hormones in urine. The kit includes a result window for displaying birth control test result and a test strip of semi rigid cotton or paper for absorbing urine.

In another aspect, a birth control monitoring device for identifying efficacy of a birth control in a human body based on a urine sample obtained from the human body is disclosed. The device includes a lightweight plastic casing extending between a first end and a second end, the casing is ergonomically designed for ease of handling, a test strip made of semi-rigid cotton or paper material (i.e., absorbent material) is attached at the second end of the casing, the test strip is configured for immersion in a urine stream for absorbing urine, a removable lid is removably engaged with the second end of the casing to protect the sterility of the test strip before use and a result window is disposed in the casing for displaying a result of the birth control check, wherein the result window is adapted to display different representations for a positive result and a negative result.

In yet another embodiment, a birth control monitoring kit is disclosed. The kit includes a test strip for absorbing urine of a human, the test strip is coupled to a flow assay strip, the flow assay strip is treated with chemicals to react with birth control hormones, the flow assay strip includes a conjugate for releasing upon contact with urine and separate detection zones for estrogen and progesterone, each detection zone comprising immobilized monoclonal antibodies specific to estrogen and progesterone, respectively.

In a further embodiment, a method for evaluating urine of a subject for monitoring the efficacy of hormonal birth control taken by the subject is described. The method includes the steps of collecting a sample of urine on a test strip, testing the urine sample with an assay strip coupled to the test strip, the assay strip includes an estrogen detection zone and a progesterone detection zone, displaying a result on a result window, wherein the result indicates the presence or absence of estrogen and progesterone. The result is positive when presence of the birth control hormones is detected and the result is negative when presence of the birth control hormones is not detected.

In yet another aspect of the present invention, the estrogen detection zone includes immobilized monoclonal anti-estrogen antibody for estrogen for detecting estrogen metabolites in the urine sample of the subject.

In another embodiment, the progesterone detection zone includes immobilized monoclonal antibody for progesterone for detecting progesterone (such as pregnanediol-3-glucuronide (PDG)) in the urine sample of the subject.

In yet another aspect, the monitoring kit includes separate assay strips for detecting estrogen and progesterone.

In another aspect, the test strip changes color to indicate the absorption of required amount of urine therein.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of the birth control monitoring device of the present invention in accordance with the disclosed architecture;

FIG. 2 illustrates an enlarged view of the result window of the personal birth control monitoring kit showing the negative result in accordance with one embodiment of the present invention;

FIG. 3 illustrates an internal structure of the personal birth control monitoring kit showing essential components in accordance with one embodiment of the present invention;

FIG. 4 illustrates an embodiment of the flow assay strip used in the personal birth control monitoring kit of the present invention; and

FIG. 5 illustrates a flow chart depicting a process of testing birth control efficacy by users using the personal birth control monitoring kit in accordance with one embodiment of the present invention.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a birth control check device that monitors levels of estrogen and progesterone in urine. There is also a long felt need in the art for a monitoring kit that enables users to check if their hormone levels are consistent with effective birth control use. Additionally, there is a long felt need in the art for a novel birth control device that offers real-time feedback to people on the efficacy of their birth control method used by them. Moreover, there is a long felt need in the art for a proactive birth control monitoring tool that enables users to potentially identify issues with their birth control regimen. Further, there is a long felt need in the art for a compact, portable, and easy-to-use device for monitoring of birth control effectiveness. Furthermore, there is a long felt need in the art for a handheld birth control device that displays results of birth control on a result window. Finally, there is a long felt need in the art for a test kit for testing the levels of birth control hormones such as estrogen and progesterone in female urine to help determine if the female is ovulating and birth control pill is ineffective.

The present invention, in one exemplary embodiment, is a method for evaluating urine of a subject for monitoring the efficacy of hormonal birth control taken by the subject. The method includes the steps of collecting a sample of urine on a test strip, testing the urine sample with an assay strip coupled to the test strip, the assay strip includes an estrogen detection zone and a progesterone detection zone, displaying a result on a result window, wherein the result indicates the presence or absence of estrogen and progesterone. The result is positive when presence of the birth control hormones is detected and the result is negative when presence of the birth control hormones is not detected.

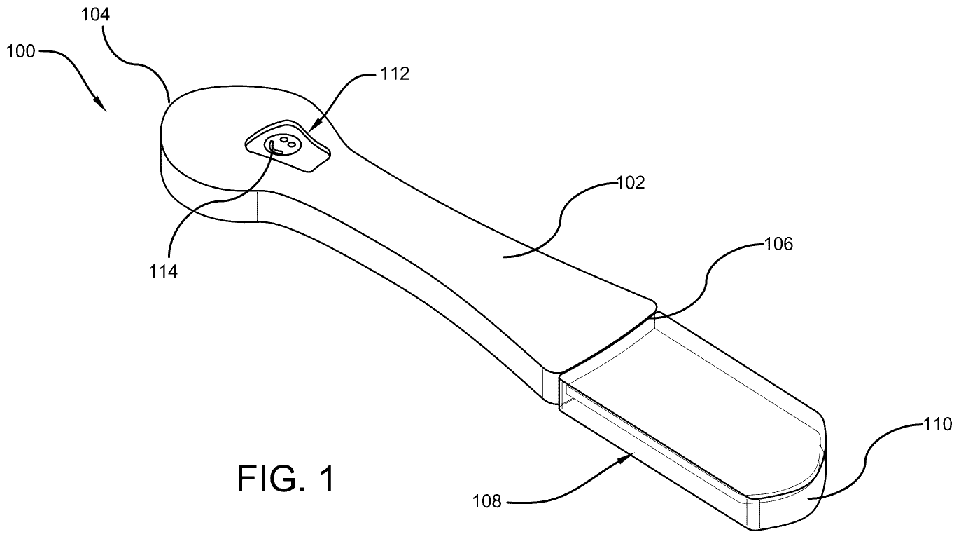

Referring initially to the drawings, FIG. 1 illustrates a perspective view of the birth control monitoring device of the present invention in accordance with the disclosed architecture. The birth control check device 100 of the present invention is designed to monitor estrogen and progesterone level in females who are using hormonal birth control methods. More specifically, the birth control check device 100 is configured to detect hormones related to birth control that are present in contraceptive pills or drugs to indicate whether birth control pills or drugs taken by a user are effective or not. The personal birth control monitoring kit 100 is a compact and portable device and includes a lightweight plastic casing 102 extending between a first end 104 and an opposite second end 106. The casing 102 is ergonomic and easy to handle, like a conventional pregnancy test kit.

At the second end 106 of the casing 100, a test urine strip 108 of semi-rigid cotton or paper material (i.e., absorbent material) is attached and extends therefrom. The urine strip 108 is adapted to be immersed in a stream of urine to absorb urine which is used by the birth control monitoring kit 100 to detect birth control hormones. The urine strip 108 is enclosed by a removable lid 110 which is removably engaged with the second end 106 of the casing 100. The removable lid 110 can be removed to expose the urine strip 108 for testing. The lid 110 protects the test strip and maintain the sterility thereof before use. In some embodiments, the color of the test urine strip 108 may change to a specific color to indicate absorption of a required quantity of urine for testing.

A result window 112 is disposed in the casing 102 for displaying a result 114 of the birth control check by the birth control monitoring kit 100. The result window 112 is adapted to display different representations for a positive result and a negative result. A positive result (as illustrated in FIG. 1) indicates presence of birth control hormones indicating birth control working fine and a negative result indicates absence of birth control hormones indicating birth control not working fine.

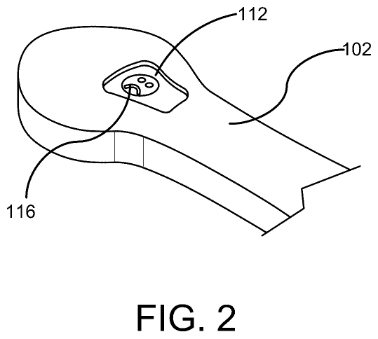

FIG. 2 illustrates an enlarged view of the result window of the personal birth control monitoring kit showing the negative result in accordance with one embodiment of the present invention. If the test urine strip 108 is immersed in urine and the urine does not contain birth control hormones such as estrogen and progesterone, then the result window 112 displays a negative result 116 indicating inefficacy of the birth control.

Preferably, the result window 112 displays the result within two minutes of immersing the test urine strip 108. Further, the device 100 is designed for one-time use and is disposed after use. In some embodiments, the test urine strip 108 can be replaced enabling the device 100 to be reused for multiple uses.

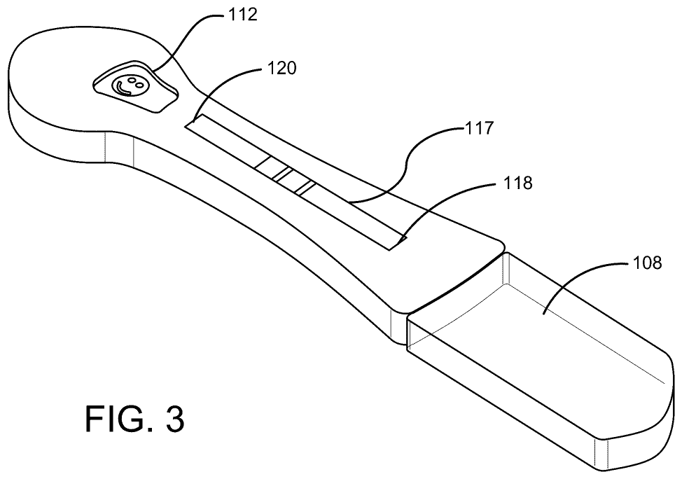

FIG. 3 illustrates a view of the personal birth control monitoring kit showing essential components in accordance with one embodiment of the present invention. The kit 100 includes a flow assay strip 117 which is coupled to the distal end 118 of the test urine strip 108. The absorbed urine passively flows to the flow assay strip 117 and the flow assay strip 117 is pre-treated with chemicals to react with birth controlling hormones. The estrogen and progesterone in the absorbed urine bind to the antibodies on the flow assay strip 117 to provide a corresponding result on the result window 112. If estrogen and progesterone are present in the urine, then a positive result is shown else a negative result is shown. The distal end 120 of the assay strip 117 is coupled to the result window 112 for displaying test result on the result window 112.

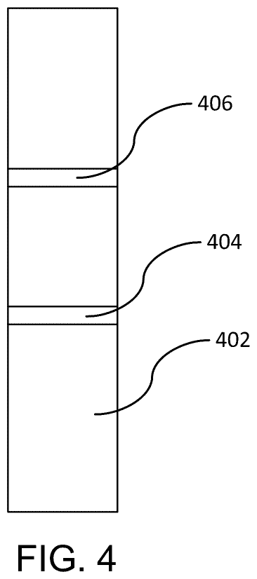

FIG. 4 illustrates an embodiment of the flow assay strip used in the personal birth control monitoring kit of the present invention. The flow assay strip 117 includes a conjugate 402 which has a dried conjugate and released upon wetting by the urine sample. In the Brønsted-Lowry definition of acids and bases, a conjugate acid-base pair consists of two substances that differ only by the presence of a proton (H⁺). A conjugate acid is formed when a proton is added to a base, and a conjugate base is formed when a proton is removed from an acid. An estrogen detection zone 404 includes immobilized monoclonal anti-estrogen antibody for estrogen for detecting estrogen metabolites (such as 17-glucuro-and sulfo-conjugates).

A progesterone detection zone 406 on the flow assay strip 117 includes immobilized monoclonal antibody for progesterone for detecting progesterone (such as pregnanediol-3-glucuronide (PDG)) in the urine sample.

In some embodiments of the present invention, separate assay strips can be used for estrogen and progesterone, wherein both the assay strips receive urine sample from the test urine strip 108.

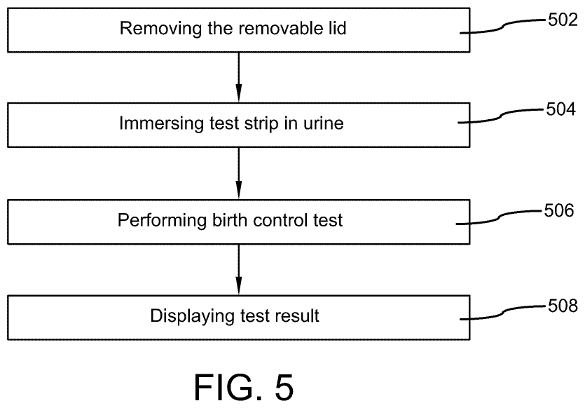

FIG. 5 illustrates a flow chart depicting a process of testing birth control efficacy by users using the personal birth control monitoring kit in accordance with one embodiment of the present invention. Initially, the removable lid 110 is removed to expose the test urine strip 108 (Step 502). Then, the test urine strip 108 is immersed in a stream of urine or dipped in urine for absorption of urine in the test urine strip 108 (Step 504). Then, the birth control test is performed by measuring estrogen level and progesterone level in the urine sample using the kit 100 (Step 506) and a corresponding result is displayed on the result window (Step 508).

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “personal birth control monitoring kit”, “birth control monitoring device”, “birth control check device”, “birth control monitoring kit”, “kit” and “device” are interchangeable and refer to the personal birth control monitoring kit 100 of the present invention.

Notwithstanding the forgoing, the personal birth control monitoring kit 100 of the present invention can be of any suitable size and configuration as is known in the art without affecting the overall concept of the invention, provided that it accomplishes the above stated objectives. One of ordinary skill in the art will appreciate that the personal birth control monitoring kit 100 as shown in the FIGS. are for illustrative purposes only, and that many other sizes and shapes of the personal birth control monitoring kit 100 are well within the scope of the present disclosure. Although the dimensions of the personal birth control monitoring kit 100 are important design parameters for user convenience, the personal birth control monitoring kit 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A birth control check device for detecting birth controlling hormones comprising:
a birth control monitoring kit;
a casing;
a urine test strip;
a flow assay strip;
a lid;
a result window;
wherein said urine test strip selectively exposable by removal of said lid;
wherein said urine test strip having an absorbent material for absorbing urine;
wherein said flow assay strip connected to a distal end of said urine test strip;
wherein said flow assay strip having a dried conjugate in an estrogen detection zone and in
a progesterone detection zone;
wherein said estrogen detection zone having an immobilized monoclonal anti-estrogen antibody for estrogen for detecting estrogen metabolites in the urine; and
further wherein said progesterone detection zone having an immobilized monoclonal antibody for progesterone for detecting progesterone in the urine.

2. The birth control check device for detecting birth controlling hormones of claim 1, wherein said immobilized monoclonal anti-estrogen antibody selected from the group consisting of a 17-glucuro conjugate and a sulfo-conjugate.

3. The birth control check device for detecting birth controlling hormones of claim 2, wherein said immobilized monoclonal antibody is a pregnanediol-3-glucuronide (PDG).

4. The birth control check device for detecting birth controlling hormones of claim 3, wherein the urine passively flows to said flow assay strip.

5. The birth control check device for detecting birth controlling hormones of claim 4, wherein said absorbent material is selected from the group consisting of a semi-rigid cotton and a paper material.

6. The birth control check device for detecting birth controlling hormones of claim 4, wherein said immobilized monoclonal anti-estrogen antibody for estrogen and said immobilized monoclonal antibody for progesterone react with birth controlling hormones in the urine.

7. The birth control check device for detecting birth controlling hormones of claim 4, wherein said immobilized monoclonal anti-estrogen antibody for estrogen and said immobilized monoclonal antibody for progesterone bind with estrogen and progesterone in the urine on said flow assay strip.

8. The birth control check device for detecting birth controlling hormones of claim 6, wherein said flow assay strip shows a positive result in said result window if estrogen and progesterone is present in the urine.

9. The birth control check device for detecting birth controlling hormones of claim 8, wherein said flow assay strip shows a negative result in said result window if estrogen and progesterone is not present in the urine.

10. The birth control check device for detecting birth controlling hormones of claim 9, wherein said flow assay strip having a distal end coupled to said result window.

11. A birth control check device for detecting birth controlling hormones comprising:
a birth control monitoring kit;
a casing;
a urine test strip;
a flow assay strip;
a lid;
a result window;
wherein said urine test strip selectively exposable by removal of said lid;
wherein said urine test strip having an absorbent material for absorbing urine;
wherein said flow assay strip connected to a distal end of said urine test strip;
wherein said flow assay strip having a dried conjugate in an estrogen detection zone and in a progesterone detection zone;
wherein said estrogen detection zone having an immobilized monoclonal anti-estrogen antibody for estrogen for detecting estrogen metabolites in the urine;
wherein said progesterone detection zone having an immobilized monoclonal antibody for progesterone for detecting progesterone in the urine;
wherein the urine passively flows to said flow assay strip; and
further wherein said immobilized monoclonal anti-estrogen antibody for estrogen and said immobilized monoclonal antibody for progesterone bind with estrogen and progesterone in the urine on said flow assay strip.

12. The birth control check device for detecting birth controlling hormones of claim 11, wherein said immobilized monoclonal anti-estrogen antibody selected from the group consisting of a 17-glucuro conjugate and a sulfo-conjugate.

13. The birth control check device for detecting birth controlling hormones of claim 12, wherein said immobilized monoclonal antibody is a pregnanediol-3-glucuronide (PDG).

14. The birth control check device for detecting birth controlling hormones of claim 13, wherein said absorbent material is selected from the group consisting of a semi-rigid cotton and a paper material.

15. The birth control check device for detecting birth controlling hormones of claim 14, wherein said flow assay strip shows a positive result in said result window if estrogen and progesterone is present in the urine.

16. The birth control check device for detecting birth controlling hormones of claim 15, wherein said flow assay strip shows a negative result in said result window if estrogen and progesterone is not present in the urine.

17. A method of detecting birth controlling hormones, the method comprising the steps of:
providing a birth control monitoring kit, a casing, a urine test strip, a flow assay strip, a lid, and a result window, wherein said flow assay strip connected to a distal end of said urine test strip, wherein said flow assay strip having a dried conjugate in an estrogen detection zone and in a progesterone detection zone, wherein said estrogen detection zone having an immobilized monoclonal anti-estrogen antibody for estrogen for detecting estrogen metabolites in the urine, further wherein said progesterone detection zone having an immobilized monoclonal antibody for progesterone for detecting progesterone in the urine;
opening said lid of said casing;
exposing said urine test strip from said casing;
urinating on said urine test strip, wherein said urine test strip having an absorbent material for absorbing urine;
flowing the urine to said flow assay strip;
binding said immobilized monoclonal anti-estrogen antibody for estrogen and said immobilized monoclonal antibody for progesterone with estrogen and progesterone in the urine on said flow assay strip; and
selectively detecting estrogen and progesterone in the urine.

18. The method of detecting birth controlling hormones of claim 17, wherein said immobilized monoclonal anti-estrogen antibody selected from the group consisting of a 17-glucuro conjugate and a sulfo-conjugate.

19. The method of detecting birth controlling hormones of claim 18, wherein said immobilized monoclonal antibody is a pregnanediol-3-glucuronide (PDG).

20. The method of detecting birth controlling hormones of claim 19 further comprising the steps of:
showing a positive result on said flow assay strip in said result window if estrogen and progesterone is detected in the urine; and
showing a negative result on said flow assay strip in said result window if estrogen and progesterone is not detected in the urine.
